(19) United States
(12) Patent Application Publication
(10) Pub. No.: US 2018/0344647 A1
(43) Pub. Date: Dec. 6, 2018

(54) PHARMACEUTICAL COMPOSITION, METHODS FOR TREATING AND USES THEREOF

(71) Applicant: Boehringer Ingelheim International GmbH, Ingelheim am Rhein (DE)

(72) Inventors: Georg BOECK, Laupheim (DE); Kerstin Julia FRANK, Ulm (DE); Venkata VOLETI, Mason, OH (US); Tracy WILLIAMSON, Cincinnati, OH (US)

(21) Appl. No.: 15/778,294

(22) PCT Filed: Dec. 1, 2016
(86) PCT No.: PCT/EP2016/079465

§ 371 Date: May 23, 2018

Related U.S. Application Data
(60) Provisional application No. 62/262,978, filed on Dec. 4, 2015.
(60) Provisional application No. 62/412,916, filed on Oct. 26, 2016.

(51) Int. Cl. A61K 9/24; A61K 31/155; A61P 3/10; A61K 9/16; A61K 9/20

(52) U.S. Cl. CPC A61K 9/209; A61K 31/155; A61P 3/10; A61K 9/167; A61K 31/7048; A61K 9/2031; A61K 9/2013; A61K 2300/00; A61K 9/2054

(57) ABSTRACT

The invention relates to solid pharmaceutical dosage forms comprising an extended release core comprising metformin hydrochloride and one or two immediate release coatings comprising linagliptin and/or empagliflozin.

### TECHNICAL FIELD OF THE INVENTION

The present invention relates to pharmaceutical compositions comprising a fixed dose combination (FDC) of the three active pharmaceutical ingredients linagliptin, empagliflozin and metformin hydrochloride wherein metformin hydrochloride is in extended release form (metformin XR); processes for the preparation thereof, and their use to treat certain diseases.

The invention also relates to a use of a pharmaceutical composition according to this invention for the manufacture of a medicament for use in a method as described hereinbefore and hereinafter.

### BACKGROUND OF THE INVENTION

Type 2 diabetes mellitus is an increasingly prevalent disease that due to a high frequency of complications leads to a significant reduction of life expectancy. Because of diabetes-associated microvascular complications, type 2 diabetes is currently the most frequent cause of adult-onset loss of vision, renal failure, and amputations in the industrialized world. In addition, the presence of type 2 diabetes mellitus is associated with a two to five fold increase in cardiovascular disease risk.

After long duration of disease, most patients with type 2 diabetes mellitus will eventually fail on oral therapy and become insulin dependent with the necessity for daily injections and multiple daily glucose measurements.

The UKPDS (United Kingdom Prospective Diabetes Study) demonstrated that intensive treatment with metformin, sulfonylureas or insulin resulted in only a limited improvement of glycemic control (difference in HbA1c ˜0.9%). In addition, even in patients within the intensive treatment arm glycemic control deteriorated significantly over time and this was attributed to deterioration of β-cell function. Importantly, intensive treatment was not associated with a significant reduction in macrovascular complications, i.e. cardiovascular events. Therefore many patients with type 2 diabetes mellitus remain inadequately treated, partly because of limitations in long term efficacy, tolerability and dosing inconvenience of existing antihyperglycemic therapies.

Oral antidiabetic drugs conventionally used in therapy (such as e.g. first- or second-line, and/or mono- or (initial or add-on) combination therapy) include, without being restricted thereto, metformin, sulphonylureas, thiazolidinediones, glinides, α-glucosidase inhibitors, DPPIV inhibitors and SGLT2 inhibitors.

The high incidence of therapeutic failure is a major contributor to the high rate of long-term hyperglycemia-associated complications or chronic damages (including micro- and macrovascular complications such as e.g. diabetic nephrophathy, retinopathy or neuropathy, or cardiovascular complications) in patients with type 2 diabetes mellitus.

Therefore, there is an unmet medical need for methods, medicaments and pharmaceutical compositions with a good efficacy with regard to glycemic control, with regard to disease-modifying properties and with regard to reduction of cardiovascular morbidity and mortality while at the same time showing an improved safety profile.

Empagliflozin is a known SGLT2 inhibitor that is described for the treatment or improvement in glycemic control in patients with type 2 diabetes mellitus, for example in WO 05/092877, WO 06/117359, WO 06/120208, WO 2010/092126, WO 2010/092123, WO 2011/039107, WO 2011/039108.

Linagliptin is a known DPPIV inhibitor that is described for the treatment or improvement in glycemic control in patients with type 2 diabetes mellitus, for example in WO2004/018468.

Dual combinations of empagliflozin or linagliptin with metformin hydrochloride as immediate release or extended release formulation are known. Formulations with a metformin hydrochloride extended release core are described for example in WO 2012/120040 and in WO 2013/131967.

### AIM OF THE PRESENT INVENTION

An aim of the present invention is to provide a solid pharmaceutical dosage form for improving glycemic control in a patient with type 2 diabetes mellitus.

Another aim of the present invention is to provide a solid pharmaceutical dosage form comprising a metformin extended release formulation, a DPPIV inhibitor and a SGLT2 inhibitor.

Another aim of the present invention is to provide a solid pharmaceutical dosage form comprising a metformin extended release formulation, linagliptin and a SGLT2 inhibitor with a sufficiently chemical stability with regard to the linagliptin content.

Another aim of the present invention is to provide a solid pharmaceutical dosage form comprising a metformin extended release formulation, linagliptin and empagliflozin with a large ratio of the amounts of metformin versus linagliptin and empagliflozin.

Another aim of the present invention is to provide a manufacturing process for the solid pharmaceutical compositions according to this invention.

Further aims of the present invention become apparent to the one skilled in the art by description hereinbefore and in the following and by the examples.

### SUMMARY OF THE INVENTION

The present invention provides a solid pharmaceutical dosage form comprising in the order from the core to the outside
a) an extended release core comprising metformin hydrochloride and one or more excipients, and
b) one or more immediate release coatings comprising linagliptin and/or empagliflozin and one or more stabilizers for stabilizing linagliptin and one or more excipients,
wherein the amount of linagliptin is in the range from 2.5 mg to 5 mg, the amount of empagliflozin is in the range from 5 mg to 25 mg and the amount of metformin hydrochloride is in the range from 500 mg to 2000 mg.

In a first aspect the present invention provides a solid pharmaceutical dosage form comprising in the order from the core to the outside
a) an extended release core comprising metformin hydrochloride and one or more excipients, and
b) none, one or more barrier coatings, and
c) an immediate release coating comprising linagliptin and empagliflozin and one or more stabilizers for stabilizing linagliptin and one or more excipients,
d) none, one or more color and/or final coatings,
wherein the amount of linagliptin is in the range from 2.5 mg to 5 mg, the amount of empagliflozin is in the range from 5 mg to 25 mg and the amount of metformin hydrochloride is in the range from 500 mg to 2000 mg.

In a second aspect the present invention provides a solid pharmaceutical dosage form comprising in the order from the core to the outside
a) an extended release core comprising metformin hydrochloride and one or more excipients, and
b) none, one or more barrier coatings, and
c) a first immediate release coating comprising one of the active ingredients linagliptin or empagliflozin and in case the active ingredient is linagliptin one or more stabilizers for stabilizing linagliptin and one or more excipients, and
d) optionally a barrier coating, and
e) a second immediate release coating comprising the other one of the active ingredients empagliflozin or linagliptin and in case the active ingredient is linagliptin one or more stabilizers for stabilizing linagliptin and one or more excipients,
f) none, one or more color and/or final coatings,
wherein the amount of linagliptin is in the range from 2.5 mg to 5 mg, the amount of empagliflozin is in the range from 5 mg to 25 mg and the amount of metformin hydrochloride is in the range from 500 mg to 2000 mg.

Within the scope of the present invention it has now been found that the solid pharmaceutical dosage forms as defined herein according to this invention have particularly advantageous properties, which make them suitable for the purpose of this invention and/or for fulfilling one or more of above needs. In particular it has been found that solid pharmaceutical dosage forms as defined herein according to this invention are sufficiently stable under various storage conditions and showing no or low degradation of linagliptin. Additionally the solid pharmaceutical dosage form described shows a dissolution of each of the active pharmaceutical ingredients metformin hydrochloride, empagliflozin and linagliptin which is in accordance with the single dosage form comprising metformin hydrochloride extended release and linagliptin immediate release and the the single dosage form comprising metformin hydrochloride extended release and empagliflozin immediate release as described for example in the WO 2013/131967.

Solid pharmaceutical dosage forms comprising metformin hydrochloride in an extended release formulation with the two active pharmaceutical ingredients linagliptin and empagliflozin in an immediate release formulation according to the invention open up new therapeutic possibilities in the improvement of glycemic control in a patient with type 2 diabetes mellitus.

According to another aspect the present invention provides a method for the treatment and/or prevention of a metabolic disease and/or a cardiovascular disease by administering a solid pharmaceutical dosage form according to the invention to a patient.

According to another aspect the present invention provides a use of a solid pharmaceutical dosage form according to this invention for the manufacture of a medicament for the treatment and/or prevention of a metabolic disease and/or a cardiovascular disease in a patient in need thereof.

According to another aspect the present invention provides a solid pharmaceutical dosage form according to this invention for use in the treatment and/or prevention of a metabolic disease and/or a cardiovascular disease in a patient in need thereof.

According to another aspect the present invention provides a method for manufacturing the solid pharmaceutical dosage forms according to the invention.

Other aspects of the invention become apparent from the description and examples.

### Definitions

The term “active ingredient” or “active pharmaceutical ingredient” or “API” of a pharmaceutical composition according to the present invention means empagliflozin or linagliptin or both empagliflozin and linagliptin. An active ingredient is also sometimes referred to herein as an “active substance”.

The term “empagliflozin” refers to the SGLT2 inhibitor 1-chloro-4-(β-D-glucopyranos-1-yl)-2-[4-((S)-tetrahydrofuran-3-yloxy)-benzyl]-benzene of the formula

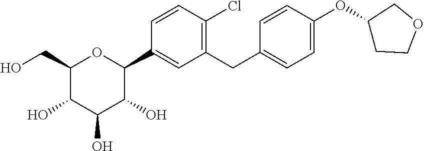 [SMILES: OC[C@H]1O[C@@H](c2ccc(Cl)c(Cc3ccc(O[C@H]4CCOC4)cc3)c2)[C@H](O)[C@@H](O)[C@@H]1O]

as described for example in WO 2005/092877. Methods of synthesis are described in the literature, for example WO 06/120208 and WO 2011/039108. According to this invention, it is to be understood that the definition of empagliflozin also comprises its hydrates, solvates and polymorphic forms thereof, and prodrugs thereof. An advantageous crystalline form of empagliflozin is described in WO 2006/117359 and WO 2011/039107 which hereby are incorporated herein in their entirety. This crystalline form possesses good solubility properties which enables a good bioavailability of the SGLT2 inhibitor. Furthermore, the crystalline form is physico-chemically stable and thus provides a good shelf-life stability of the pharmaceutical composition. Preferred pharmaceutical compositions, such as solid formulations for oral administration, for example tablets, are described in WO 2010/092126, which hereby is incorporated herein in its entirety.

The term “linagliptin” as employed herein refers to linagliptin, a pharmaceutically acceptable salt thereof, a hydrate or solvate thereof, or a polymorphic form thereof. Crystalline forms are described in WO 2007/128721. Preferred crystalline forms are the polymorphs A and B described therein. In particular, linagliptin is the free base 1-[(4-methyl-quinazolin-2-yl)methyl]-3-methyl-7-(2-butyn-1-yl)-8-(3-(R)-amino-piperidin-1-yl)-xanthine. As linagliptin or a pharmaceutically acceptable salt thereof, linagliptin is preferred. Methods for the manufacture of linagliptin are described in the patent applications WO 2004/018468 and WO 2006/048427 for example. A therapeutic dose of linagliptin may be 5 mg per patient per day. 1-[(4-Methyl-quinazolin-2-yl)methyl]-3-methyl-7-(2-butyn-1-yl)-8-(3-(R)-amino-piperidin-1-yl)-xanthine (linagliptin):

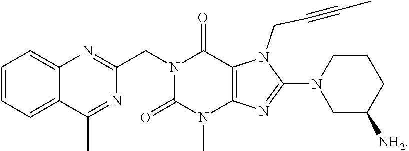 [SMILES: CC#CCn1c(N2CCC[C@@H](N)C2)nc2c1c(=O)n(Cc1nc(C)c3ccccc3n1)c(=O)n2C]

The term “pre-diabetes” is the condition wherein an individual is pre-disposed to the development of type 2 diabetes. Pre-diabetes extends the definition of impaired glucose tolerance to include individuals with a fasting blood glucose within the high normal range ≥100 mg/dL (J. B. Meigs, et al. Diabetes 2003; 52:1475-1484) and fasting hyperinsulinemia (elevated plasma insulin concentration). The scientific and medical basis for identifying pre-diabetes as a serious health threat is laid out in a Position Statement entitled “The Prevention or Delay of Type 2 Diabetes” issued jointly by the American Diabetes Association and the National Institute of Diabetes and Digestive and Kidney Diseases (Diabetes Care 2002; 25:742-749).

The term “type 2 diabetes mellitus” or “T2DM” is defined as the condition in which a subject has a fasting blood glucose or serum glucose concentration greater than 125 mg/dL (6.94 mmol/L). The measurement of blood glucose values is a standard procedure in routine medical analysis. If a glucose tolerance test is carried out, the blood sugar level of a diabetic will be in excess of 200 mg of glucose per dL (11.1 mmol/l) of plasma 2 hours after 75 g of glucose have been taken on an empty stomach. In a glucose tolerance test 75 g of glucose are administered orally to the patient being tested after 10-12 hours of fasting and the blood sugar level is recorded immediately before taking the glucose and 1 and 2 hours after taking it. In a healthy subject, the blood sugar level before taking the glucose will be between 60 and 110 mg per dL of plasma, less than 200 mg per dL 1 hour after taking the glucose and less than 140 mg per dL after 2 hours. If after 2 hours the value is between 140 and 200 mg, this is regarded as abnormal glucose tolerance.

The term “late stage type 2 diabetes mellitus” includes patients with a secondary drug failure, indication for insulin therapy and progression to micro- and macrovascular complications e.g. diabetic nephropathy, or coronary heart disease (CHD).

The term “HbA1c” refers to the product of a non-enzymatic glycation of the haemoglobin B chain. Its determination is well known to one skilled in the art. In monitoring the treatment of diabetes mellitus the HbA1c value is of exceptional importance. As its production depends essentially on the blood sugar level and the life of the erythrocytes, the HbA1c in the sense of a “blood sugar memory” reflects the average blood sugar levels of the preceding 4-6 weeks. Diabetic patients whose HbA1c value is consistently well adjusted by intensive diabetes treatment (i.e. <6.5% of the total haemoglobin in the sample), are significantly better protected against diabetic microangiopathy. For example, metformin on its own achieves an average improvement in the HbA1c value in the diabetic of the order of 1.0-1.5%. This reduction of the HbA1C value is not sufficient in all diabetics to achieve the desired target range of <6.5% and preferably <6% HbA1c.

The term “insufficient glycemic control” or “inadequate glycemic control” in the scope of the present invention means a condition wherein patients show HbA1c values above 6.5%, in particular above 7.0%, even more preferably above 7.5%, especially above 8%.

The “metabolic syndrome”, also called “syndrome X” (when used in the context of a metabolic disorder), also called the “dysmetabolic syndrome” is a syndrome complex with the cardinal feature being insulin resistance (Laaksonen D E, et al. Am J Epidemiol 2002; 156:1070-7). According to the ATP III/NCEP guidelines (Executive Summary of the Third Report of the National Cholesterol Education Program (NCEP) Expert Panel on Detection, Evaluation, and Treatment of High Blood Cholesterol in Adults (Adult Treatment Panel III) JAMA: Journal of the American Medical Association (2001) 285:2486-2497), diagnosis of the metabolic syndrome is made when three or more of the following risk factors are present:
1. Abdominal obesity, defined as waist circumference >40 inches or 102 cm in men, and >35 inches or 94 cm in women; or with regard to a Japanese ethnicity or Japanese patients defined as waist circumference ≥85 cm in men and ≥90 cm in women;
2. Triglycerides: ≥150 mg/dL
3. HDL-cholesterol <40 mg/dL in men
4. Blood pressure ≥130/85 mm Hg (SBP ≥130 or DBP ≥85)
5. Fasting blood glucose ≥100 mg/dL

The NCEP definitions have been validated (Laaksonen D E, et al. Am J Epidemiol. (2002) 156:1070-7). Triglycerides and HDL cholesterol in the blood can also be determined by standard methods in medical analysis and are described for example in Thomas L (Editor): “Labor and Diagnose”, TH-Books Verlagsgesellschaft mbH, Frankfurt/Main, 2000.

According to a commonly used definition, hypertension is diagnosed if the systolic blood pressure (SBP) exceeds a value of 140 mm Hg and diastolic blood pressure (DBP) exceeds a value of 90 mm Hg. If a patient is suffering from manifest diabetes it is currently recommended that the systolic blood pressure be reduced to a level below 130 mm Hg and the diastolic blood pressure be lowered to below 80 mm Hg.

The terms “treatment” and “treating” comprise therapeutic treatment of patients having already developed said condition, in particular in manifest form. Therapeutic treatment may be symptomatic treatment in order to relieve the symptoms of the specific indication or causal treatment in order to reverse or partially reverse the conditions of the indication or to stop or slow down progression of the disease. Thus the compositions and methods of the present invention may be used for instance as therapeutic treatment over a period of time as well as for chronic therapy.

The terms “prophylactically treating”, “preventivally treating” and “preventing” are used interchangeably and comprise a treatment of patients at risk to develop a condition mentioned hereinbefore, thus reducing said risk.

The term “tablet” comprises tablets without a coating and tablets with one or more coatings. Furthermore the “term” tablet comprises tablets having one, two, three or even more layers and press-coated tablets, wherein each of the beforementioned types of tablets may be without or with one or more coatings. The term “tablet” also comprises mini, melt, chewable, effervescent and orally disintegrating tablets.

The terms “pharmacopoe” and “pharmacopoeias” refer to standard pharmacopoeias such as the “USP 31-NF 26 through Second Supplement” (United States Pharmacopeial Convention) or the “European Pharmacopoeia 6.3” (European Directorate for the Quality of Medicines and Health Care, 2000-2009).

### DETAILED DESCRIPTION

According to the present invention the solid pharmaceutical dosage form preferably comprises one or more barrier coatings between the extended release core and the one or more immediate release coating(s).

According to the present invention the solid pharmaceutical dosage form preferably further compises one or more color and/or final coatings on the outside of the one or more immediate release coating(s).

The first aspect of the invention relates to a solid pharmaceutical dosage form comprising in the order from the core to the outside
a) an extended release core comprising metformin hydrochloride and one or more excipients, and
b) none, one or more barrier coatings, and
c) an immediate release coating comprising linagliptin and empagliflozin and one or more stabilizers for stabilizing linagliptin and one or more excipients,
d) none, one or more color and/or final coatings,
wherein the amount of linagliptin is in the range from 2.5 mg to 5 mg, the amount of empagliflozin is in the range from 5 mg to 25 mg and the amount of metformin hydrochloride is in the range from 500 mg to 2000 mg.

The second aspect of the invention relates to a solid pharmaceutical dosage form comprising in the order from the core to the outside
a) an extended release core comprising metformin hydrochloride and one or more excipients, and
b) none, one or more barrier coatings, and
c) a first immediate release coating comprising one or the active ingredients linagliptin or empagliflozin and in case the active ingredient is linagliptin one or more stabilizers for stabilizing linagliptin and one or more excipients, and
d) optionally a barrier coating, and
e) a second immediate release coating comprising the other one of the active ingredients empagliflozin or linagliptin and in case the active ingredient is linagliptin one or more stabilizers for stabilizing linagliptin and one or more excipients,
f) none, one or more color and/or final coatings,
wherein the amount of linagliptin is in the range from 2.5 mg to 5 mg, the amount of empagliflozin is in the range from 5 mg to 25 mg and the amount of metformin hydrochloride is in the range from 500 mg to 2000 mg.

According to a preferred embodiment of the first aspect the invention relates to a solid pharmaceutical dosage form comprising in the order from the core to the outside
a) an extended release core comprising metformin hydrochloride and one or more excipients, and
b) one or more barrier coatings, and
c) an immediate release coating comprising linagliptin and empagliflozin and one or more stabilizers for stabilizing linagliptin and one or more excipients,
d) one or more color and/or final coatings,
wherein the amount of linagliptin is in the range from 2.5 mg to 5 mg, the amount of empagliflozin is in the range from 5 mg to 25 mg and the amount of metformin hydrochloride is in the range from 500 mg to 2000 mg.

According to a preferred variant of the second aspect the invention relates to a solid pharmaceutical dosage form comprising in the order from the core to the outside
a) an extended release core comprising metformin hydrochloride and one or more excipients, and
b) one or more barrier coatings, and
c) a first immediate release coating comprising one or the active ingredients linagliptin or empagliflozin and in case the active ingredient is linagliptin one or more stabilizers for stabilizing linagliptin and one or more excipients, and
d) optionally a barrier coating, and
e) a second immediate release coating comprising the other one of the active ingredients empagliflozin or linagliptin and in case the active ingredient is linagliptin one or more stabilizers for stabilizing linagliptin and one or more excipients,
f) one or more color and/or final coatings,
wherein the amount of linagliptin is in the range from 2.5 mg to 5 mg, the amount of empagliflozin is in the range from 5 mg to 25 mg and the amount of metformin hydrochloride is in the range from 500 mg to 2000 mg.

The extended-release mechanism for the metformin hydrochloride core may be based on an expandable polymeric swelling formulation that increases gastric retention and extends drug release from the matrix.

Exemplary extended release formulations of metformin are disclosed in U.S. Pat. No. 6,340,475; U.S. Pat. No. 6,488,962; U.S. Pat. No. 6,635,280; U.S. Pat. No. 6,723,340; U.S. Pat. No. 7,780,987; U.S. Pat. No. 6,866,866; U.S. Pat. No. 6,495,162; U.S. Pat. No. 6,790,459; U.S. Pat. No. 6,866,866; U.S. Pat. No. 6,475,521; and U.S. Pat. No. 6,660,300; the disclosures of which are incorporated herein in their entireties.

A particular extended release formulation of metformin is described in U.S. Pat. No. 6,723,340, the disclosure of which is incorporated herein in its entirety.

In an embodiment, the extended release core comprises a matrix formulation with metformin hydrochloride dispersed therein, said matrix formulation comprising one or more extended release materials. For example the matrix formulation is compressed into a tablet form.

According to an embodiment of the present invention the extended release core comprises metformin hydrochloride, one or more swellable and/or extended release materials, and one or more further excipients.

For example the swellable and/or extended release material comprises polyethylene oxide and hydroxypropyl methylcellulose. A synonym of hydroxypropyl methylcellulose is hypromellose.

In a particular embodiment, the extended release core comprises metformin hydrochloride, hydroxypropyl methylcellulose (hypromellose), polyethylene oxide, microcrystalline cellulose, and magnesium stearate.

In another particular embodiment, the extended release core comprises metformin hydrochloride, polyethylene oxide, low molecular weight hydroxypropyl methylcellulose (hypromellose, e.g. Methocel E5), and magnesium stearate.

A particular extended release formulation of metformin is described in U.S. Pat. No. 6,723,340 as follows:

In an embodiment, the extended release material comprises poly(ethylene oxide) and/or hydroxypropyl methylcellulose (HPMC), preferably a combination of poly(ethylene oxide) and hydroxypropyl methylcellulose (HPMC), preferably at a weight ratio that causes the formulation or matrix to swell upon contact with gastric fluid to a size large enough to provide gastric retention.

The poly(ethylene oxide) component of the matrix may limit initial release of the drug and may impart gastric retention through swelling. The hydroxypropyl methylcellulose (HPMC) component may lower the amount of poly(ethylene oxide) required while still allowing the swelling to occur.

Preferably, the poly(ethylene oxide) has a viscosity average molecular weight of from about 2,000,000 to about 10,000,000 daltons, more preferably from about 4,000,000 to about 7,000,000 daltons.

Preferably, the hydroxypropyl methylcellulose (HPMC) has a viscosity of from about 4,000 centipoise to about 200,000 centipoise, more preferably from about 50,000 to about 200,000 centipoise, even more preferably 80,000 centipoise to about 120,000 centipoise, measured as a 2% solution in water.

More preferably, the poly(ethylene oxide) has a viscosity average molecular weight of from about 4,000,000 to about 7,000,000 daltons, and the hydroxypropyl methylcellulose (HPMC) has a viscosity of from about 80,000 centipoise to about 120,000 centipoise, measured as a 2% solution in water.

In an embodiment, the weight ratio of the poly(ethylene oxide) to hydroxypropyl methylcellulose (HPMC) is within the range from about 1:3 to 3:1, preferably 1:2 to 2:1.

In a further embodiment, the weight ratio of the poly(ethylene oxide) and hydroxypropyl methylcellulose (HPMC) in combination constitutes from about 15% to about 90%, or from about 30% to about 65%, or from about 40% to about 50%, by weight of the metformin part.

Extended release cores in accordance with this invention can be prepared by common tabletting methods that involve mixing, comminution, and fabrication steps commonly practiced by and well known to those skilled in the art of manufacturing drug formulations. Examples of such techniques are:
(1) Direct compression using appropriate punches and dies, typically fitted to a suitable rotary tabletting press;
(2) Injection or compression molding;
(3) Granulation by fluid bed, by low or high shear granulation, or by roller compaction, followed by compression; and
(4) Extrusion of a paste into a mold or to an extrudate to be cut into lengths.

When tablets are made by direct compression, the addition of lubricants may be helpful and is sometimes important to promote powder flow and to prevent breaking of the tablet when the pressure is relieved. Examples of typical lubricants are magnesium stearate (in a concentration of from 0.25% to 3% by weight, preferably about 1% or less by weight, in the powder mix), stearic acid (0.5% to 3% by weight), and hydrogenated vegetable oil (preferably hydrogenated and refined triglycerides of stearic and palmitic acids at about 1% to 5% by weight, most preferably about 2% by weight).

Additional excipients may be added, such as e.g. granulating aids (e.g. low molecular weight HPMC at 2-5% by weight), binders (e.g. microcrystalline cellulose), and additives to enhance powder flowability, tablet hardness, and tablet friability and to reduce adherence to the die wall.

In an embodiment of this invention the extended release core is a tablet.

An exemplary extended release core comprises metformin hydrochloride, a combination of poly(ethylene oxide) and hydroxypropyl methylcellulose (e.g. Methocel K100M) as a matrix for a swellable extended release tablet, microcrystalline cellulose as binder, low molecular weight hydroxypropyl methylcellulose (e.g. Methocel E5) as granulating aid, and magnesium stearate as lubricant.

The composition of a representative extended release core is provided as follows:
metformin hydrochloride, e.g. 49.97% by weight of the extended release core,
poly(ethylene oxide), e.g. 26.50% by weight of the extended release core,
hydroxypropyl methylcellulose (e.g. Methocel K100M), e.g. 16.08% by weight of the first part, microcrystalline cellulose, e.g. 4.99% by weight of the extended release core,
low molecular weight hydroxypropyl methylcellulose (e.g. Methocel E5), e.g. 1.70% by weight of the extended release core, and
magnesium stearate, e.g. 0.75% by weight of the extended release core.

Tablets may be formulated by dry blending and preparation of a granulate comprising metformin hydrochloride and low molecular weight HPMC (e.g. Methocel E5) and the remaining excipients listed above, followed by pressing on a tablet press.

Such an extended release core of metformin is disclosed in U.S. Pat. No. 6,723,340 (e.g. Example 3), the disclosure of which is incorporated herein in its entirety.

In an embodiment, the amount of metformin hydrochloride is e.g. 500 mg, 750 mg or 1000 mg metformin hydrochloride.

As further example of a lubricant sodium stearyl fumarate may be mentioned (e.g. at about 0.25-3%by weight).

Another particular extended release core comprises metformin hydrochloride (e.g. 750 mg or 1000 mg), poly(ethylene oxide), low molecular weight hydroxypropyl methylcellulose (hypromellose, e.g. Methocel E5), and magnesium stearate.

In a certain embodiment, the poly(ethylene oxide) as swelling and/or extended release material has an approximate molecular weight of 7,000,000, and/or a viscosity range 7500-10,000 centipoise measured as aqueous 1% solution at 25° C.

In a preferred embodiment, the poly(ethylene oxide) is WSR-303 NF TG LEO or SENTRY™ POLYOX™ WSR 303.

In a certain embodiment, the low molecular weight hydroxypropyl methylcellulose as (granulation) binder is hypromellose having viscosity of 5 centipoise measured as aqueous 2% solution at 20° C.

In a preferred embodiment, the low molecular weight hydroxypropyl methylcellulose is HPMC 2910-5 or Methocel E5.

The composition of a representative extended release core is provided as follows:
metformin hydrochloride, e.g. 750.0 mg, 65.68% by weight of the extended release core,
poly(ethylene oxide) (e.g. WSR-303 NF TG LEO or SENTRY™ POLYOX™ WSR 303), e.g. 355 mg, 31% by weight of the extended release core,
low molecular weight hydroxypropyl methylcellulose (e.g. HPMC 2910-5 or Methocel E5), e.g. 25.5 mg, 2.23% by weight of the extended release core, and
magnesium stearate, e.g. 11.3 mg, 0.99% by weight of the extended release core.

The composition of another representative extended release core is provided as follows:
metformin hydrochloride, e.g. 1000.0 mg, 70.5% by weight of the extended release core,
poly(ethylene oxide) (e.g. WSR-303 NF TG LEO), e.g. 370 mg, 26% by weight of the extended release core, low molecular weight hydroxypropyl methylcellulose (e.g. HPMC 2910-5 or Methocel E5), e.g. 34 mg, 2.4% by weight of the extended release core, and magnesium stearate, e.g. 15 mg, 1% by weight of the extended release core.

According to an embodiment of this invention the manufacturing of extended release cores comprises metformin wet granulation (e.g. high shear granulation) in the presence of low molecular weight HPMC (hypromellose, e.g. HPMC 2910-5 or Methocel E5) as binder for granulation, followed by optional wet milling, drying (e.g. fluid bed drying, e.g. <0.2% LOD) and milling (e.g. dry granulation milling). The milled granulates are subsequently used for blending with poly(ethylene oxide) (e.g. WSR-303 NF TG LEO or SENTRY™ POLYOX™ WSR 303), then magnesium stearate as lubricant for tableting is added for final blending. The obtained metformin blend is used for compression of single layer tablets.

In the metformin granulation process the metformin hydrochloride (750 mg or 1000 mg, 96.7% by weight of total dry granulate) and part of hypromellose (e.g. HPMC 2910-5 or Methocel E5, 2.4% by weight of total dry granulate) are premixed and granulated with an aqueous (10% w/w) binder solution of remaining part of hypromellose (e.g. HPMC 2910-5 or Methocel E5, 0.90% by weight of total dry granulate) using a high shear granulator.

Before being used in the manufacturing process, the excipients during dispensing may be pre-sieved and the metformin hydrochloride may be pre-milled.

In a further embodiment, the extended release core allows for targeted, controlled delivery of metformin to the upper gastrointestinal (GI) tract. In a further embodiment, the metformin extended release core is a hydrogel matrix system and contains a swelling hydrophilic polymer and further excipients, which may allow the metformin tablet core to be retained in the stomach (‘gastric retention’) for approximately eight to nine hours. During this time, the tablet core's metformin is steadily delivered to the upper GI tract at the desired rate and time, without potentially irritating ‘burst’ of drug. This gradual, extended release typically allows for more of the metformin drug to be absorbed in the upper GI tract and minimizes the amount of drug that passes through to the lower GI tract.

In another embodiment, the extended release (e.g. 750 mg or 1000 mg) cores of this invention provide gastric retention with extended dose of metformin released over approximately 12 hours.

According to the first and second aspect of the invention there is one or more barrier coatings between the extended release core and an immediate release coating.

The metformin XR core is barrier coated using one or more barrier coating agents (and optionally a plasticizer and optionally futher excipients), such as with a mixture of hydroxypropylcellulose and hydroxypropyl methylcellulose, a mixture of polyvinyl alcohol (PVA) and polyethylene glycol (PEG), a mixture of hydroxypropyl methylcellulose and either polyethylene glycol (PEG) or propylene glycol (PG), or any other suitable immediate-release film-coating agent(s). A commercial film-coat is Opadry®, Opadry II® or other Opardy IR film coat, which are formulated powder blend provided by Colorcon. Optionally the barrier coat may further comprise a glidant.

For example, a barrier coat according to this invention may comprise or consist essentially of a film-coating agent which is hydroxypropyl methylcellulose (HPMC, hypromellose such as e.g. Methocel E5, Pharmacoat 606 or HPMC 2910), hydroxypropyl cellulose, and a glidant which is talc, and optionally one or more pigments and/or colors, for example titanium dioxide.

In an embodiment, the barrier coat may be from 3% to 7% w/w (e.g. 5%) per weight of the total/metformin HCl/linagliptin/empagliflozin containing composition. Preferably, the barrier coat may have a weight in the range from 50 to 100 mg, for example 60-75 mg (such as for the formulations containing 750 mg metformin HCl) or 75-95 mg (such as for the formulations containing 1000 mg metformin HCl).

The barrier coat separates the metformin XR core from the API-containing film coat. Typically, for the preparation of film-coated tablets a coating suspension is prepared and the metformin HCl containing tablet cores are coated with the barrier coating suspension using standard film coater. The film coating solvent is a volatile component, which does not remain in the final product.

The solid pharmaceutical dosage form according to the invention comprises one or more stabilizers for stabilizing linagliptin. Suitable stabilizers are for example a basic and/or nucleophilic excipient, preferably L-arginine.

According to the first aspect of the invention there is an immediate release coating comprising linagliptin and empagliflozin and one or more stabilizers for stabilizing linagliptin and one or more excipients.

In an embodiment of this first aspect of the invention, the immediate release coating comprises a film coat formulation containing linagliptin and empagliflozin as active pharmaceutical ingredients, said film coat formulation comprising linagliptin, empagliflozin, a stabilizer for stabilizing linagliptin (e.g. a basic and/or nucleophilic excipient, preferably L-arginine as stabilizer), one or more film-coating agents (such as e.g. hydroxypropyl methylcellulose, e.g. Hypromellose 2910, Methocel E5, or Methocel E15), a plasticizer (such as e.g. polyethylene glycol, e.g. Macrogol 400, 6000 or 8000, or propylene glycol), and, optionally, a glidant and/or anti-tacking agent (such as e.g. talc).

In an embodiment, the weight ratio of the L-arginine to linagliptin is within the range from about 20:1 to about 1:1, or from about 15:1 to about 1:1, or from about 10:1 to about 1:1, or from about 8:1 to about 1:1, or from about 8:1 to about 2:1, or from about 6:1 to about 2:1, especially about 4:1.

In a preferred embodiment, the weight ratio of the L-arginine to linagliptin is within the range from about 4:1 to about 2:1, especially about 4:1.

For example, the composition of a representative immediate release coating is provided as follows:
polyethylene glycol (e.g. Macrogol 400, 6000 or 8000, in particular Macrogol 6000), e.g. from about 6 to about 14 mg, especially 8-12 mg, in particular 10 mg;
linagliptin, e.g. 2.5 mg or 5 mg;
empagliflozin, e.g. 5 mg, 10 mg, 12.5 mg or 25 mg;
L-arginine, e.g. depending from need of stabilizer amount, e.g. in the range from about 5 mg to about 15 mg (e.g. 10 mg), or up to about 15 mg to about 25 mg (e.g. 20 mg);
talc, e.g. from about 0 mg to about 15 mg or 6 to 12 mg (especially 9 mg).
a film-coating agent, such as e.g. hydroxypropyl methylcellulose (e.g. Methocel E5, Methocel E15, or Pharmacoat 603 or 606 or HPMC 2910 Hypromellose), e.g. from about 30 mg to about 46 mg (especially from 35 mg to 43 mg, for example 38 mg);
a plasticizer, such as polyethylene glycol (e.g. Macrogol 400, 6000 or 8000, in particular Macrogol 8000), e.g. from about 0 to about 10 mg, especially 1-5 mg, in particular 2 mg; or propylene glycol, e.g. from about 0 mg to about 15 mg (especially 9 mg or 0 mg); and

In certain embodiments, the hydroxypropyl methylcellulose (HPMC) as film-coating agent is hypromellose having viscosity from about 3 centipoise to about 15 centipoise or up to 40-60 centipoise, measured as aqueous 2% solution at 20° C., e.g. 3, 5, 6, 15 or 50 cP, such as e.g. hypromellose 2910 with nominal viscosity of 3 cP (HPMC 2910-3), hypromellose 2910 with nominal viscosity of 5 cP (HPMC 2910-5), hypromellose 2910 with nominal viscosity of 6 cP (HPMC 2910-6), hypromellose 2910 with nominal viscosity of 15 cP (HPMC 2910-15), hypromellose 2910 with nominal viscosity of 50 cP (HPMC 2910-50), Methocel E5, Methocel E15, Pharmacoat 603, Pharmacoat 606, or Pharmacoat 615.

In certain embodiments, the polyethylene glycol (PEG) as plasticizer is macrogol having average molecular weight from about 400 to about 8000 daltons, e.g. 400, 1500, 3000, 4000, 6000 or 8000 D, such as e.g. Marcrogol 400, Marcrogol 6000 or Marcrogol 8000.

In certain embodiments, the coating material for API coating is commercially available under the trade name Opadry®, Opadry II® or other Opadry® film coat.

The following table contains examples IR.1 to IR.4 of compositions of immediate release coatings according to the first embodiment of the present invention:

TABLE 1
Example formulations for API containing
immediate release coatings

| | IR.1 | IR.2 | IR.3 | IR.4 |
|---|---|---|---|---|
| | Amount | Amount | Amount | Amount |
| Ingredient | in mg | in mg | in mg | in mg |
| Polyethylene | 10 | 10 | 10 | 10 |
| glycol (6000 | | | | |
| USP/NF) | | | | |
| L-Arginine | 20 | 20 | 10 | 10 |
| Linagliptin | 5 | 5 | 2.5 | 2.5 |
| Empagliflozin | 10 | 25 | 5 | 12.5 |
| Talc | 9 | 9 | 9 | 9 |
| Hydroxypropyl- | 38 | 38 | 38 | 38 |
| methylcellulose | | | | |
| (HPMC 2910, 3 | | | | |
| cP) | | | | |
| Polyethylene | 2 | 2 | 2 | 2 |
| glycol (8000 | | | | |
| USP/NF) | | | | |

The composition of a representative linagliptin and empagliflozin containing film coat suspension further comprises water, e.g. from about 240 mg to about 1440 mg, especially in the range from 608 mg to 1150 mg. The total solids concentration of the suspension is from about 4% to about 12.5% w/w, especially from 4% to 8% w/w.

The sum solids of the linagliptin and empagliflozin coating suspension is from about 50 mg to about 140 mg, preferably from about 70 mg to about 120 mg. For example, the sum solids is about 76.5 mg of solid amount of the film coating suspension for 2.5 mg linagliptin and 5 mg empagliflozin, and 109 mg sum solid amount of the film coating suspension for 5 mg linagliptin and 25 mg empagliflozin. To keep the solid concentration in the coating suspension constant, the amount of water is adjusted.

Preferably the pharmaceutical composition according to this embodiment additionally comprises one or more optional color and/or final coatings.

Film coating suspensions/solutions of API (linagliptin and/or empagliflozin) according to this invention can be prepared by common methods, such as follows:

The film-coating agent hydroxypropyl methylcellulose (HPMC), the plasticizer polyethylene glycol (PEG) (e.g. Macrogol 400, 6000 or 8000) or, as alternative plasticizer, propylene glycol (PG) and water are dissolved and mixed by a suitable mixer (e.g. by propeller mixer) to produce the API-free coating solution. Optionally, the glidant talc suspended in water is added and the obtained suspension is homogenized. Talc may be used optionally. The active ingredients linagliptin and empagliflozin and the stabilizer L-arginine are dissolved or suspended in water and added to the aqueous solution of HPMC, PEG or PG, and, optional talc, and dispersed by a suitable mixer (e.g. by propeller mixer) to provide the API coating suspension. The order of the compounds in the dissolving or suspending and addition procedure is given as an example and may be altered.

Alternatively, the film-coating agent hydroxypropyl methylcellulose (HPMC) and water are dissolved and mixed by a suitable mixer (e.g. by Ultraturrax).

The stabilizer L-arginine, the plasticizer polyethylene glycol (PEG) (e.g. Macrogol 400, 6000 or 8000) or propylene glycol (PG), optional talc, and water are dispersed, e.g. by homogenization using e.g. ultra turrax.

After degassing of the HPMC solution (or directly after manufacturing of the HPMC solution), the aqueous suspension of PEG or PG, optional L-arginine and optional talc are added to the aqueous HPMC solution and mixed/homogenized.

The APIs linagliptin and empagliflozin are dissolved or suspended in water and added to the aqueous solution of HPMC, PEG or PG, optional L-arginine and optional talc to provide the API coating suspension.

The film-coating operation is carried out in a conventional film coater. The film coating suspensions/solutions are coated at barrier coated metformin XR cores via coating process. Preliminary preheating of the cores may be necessary, due to need of equilibrium of water amount of the cores.

The spray rate and air flow through the coating pan is adjusted to produce a uniform coating and coverage of the entire width of the tablet bed. The amount of the coating suspension applied can be controlled by percent weight gain of tablet cores and typically ranges from about 1 to about 10%.

In one aspect, this range results in linagliptin drug assay close to the desired 2.5 mg or 5 mg with a standard deviation of about 2-4% for content uniformity assay of linagliptin. In another aspect, this range results in empagliflozin drug assay close to the desired 5 mg, 12.5 mg, 10 mg or 25 mg with a standard deviation of about 2-4% for content uniformity assay of empagliflozin. The duration of the coating step is about 4-10 hours. The duration of the coating step depends on batch size, process parameters like spray rate and solid concentrations of the coating suspension.

The API coating suspension is applied to the barrier coated tablet cores containing the metformin XR formulation and the amount of solids deposited in the API film layer is controlled to achieve the desired API doses.

The weight of the cores and film coated tablets may be controlled by total amount sprayed or percent weight gain during the coating process. Instead of or in addition to weight gain method a PAT method, e.g. online NIR or Raman method for end point detection of assay of API may be used.

Preferably the pharmaceutical dosage form according to this embodiment additionally comprises one or more color and/or final coatings. For example in addition to the extended release core, the barrier coating and the immediate release coating, the solid pharmaceutical dosage forms comprise a color coat and a clear coat and optionally a wax, such asw carnauba wax.

The final solid pharmaceutical dosage form of the present invention is preferably a tablet. Such tablets may be further film-coated with a color and/or final film over-coat, such as with a mixture of hydroxypropylcellulose and hydroxypropyl methylcellulose containing titanium dioxide and/or other coloring agents, such as iron oxides, dyes, and lakes; a mixture of polyvinyl alcohol (PVA) and polyethylene glycol (PEG) containing titanium dioxide and/or other coloring agents, such as iron oxides, dyes, and lakes; a mixture of hydroxypropyl methylcellulose and either polyethylene glycol (PEG) or propylene glycol (PG) containing titanium dioxide and/or other coloring agents, such as iron oxides, dyes, and lakes; or any other suitable immediate-release film-coating agent(s). The coat may provide taste masking and additional stability to the final tablet. A commercial film-coat is Opadry®, Opadry II® or other Opardy IR film coat, which are formulated powder blend provided by Colorcon.

Preferably, for the preparation of film-coated tablets a coating suspension is prepared and the tablet cores are coated with the coating suspension. Typically for the API-free color-coat to a weight gain of about 2-4%, preferably about 3%, using standard film coater. The film coating solvent is a volatile component, which does not remain in the final product. A typical film-coat comprise a film coating agent, a plasticizer, and, optionally, a glidant, one or more pigments and/or colors. For example, the film coat may comprise hydroxypropylmethylcellulose (HPMC), propylene glycol or polyethylene glycol, talc and, optionally, titanium dioxide and/or iron oxide (e.g. iron oxide yellow and/or red and/or black).

In further embodiments of the film coating layers of this invention, the film-coating agent may be one or more of hydroxypropyl methylcellulose (HPMC, hypromellose such as e.g. hypromellose 2910 with nominal viscosity of 3, 5, 6, 15 and/or 50 cP, Methocel E5, Methocel E15, Pharmacoat 603, Pharmacoat 606, and/or Pharmacoat 615), polyvinyl alcohol (PVA), ethyl cellulose, hydroxypropyl cellulose, methacrylic and/or acrylic polymer, or mixtures thereof (e.g. a mixture of one or more types of HPMC).

According to an embodiment of this invention a clear coat is formed on top of the color coated tablet.

The solid pharmaceutical dosage form according to this invention may be packaged in a variety of ways. Generally, an article for distribution includes a container that contains the pharmaceutical dosage form in an appropriate form. Tablets are typically packed in an appropriate primary package for easy handling, distribution and storage and for assurance of proper stability of the composition at prolonged contact with the environment during storage. Primary containers for tablets may be bottles or blister packs, optionally with desiccant.

A suitable bottle may be made from glass or polymer (preferably polypropylene (PP) or high density polyethylene (HD-PE)) and sealed with a screw cap. The screw cap may be provided with a child resistant safety closure (e.g. press-and-twist closure) for preventing or hampering access to the contents by children. If required (e.g. in regions with high humidity), by the additional use of a desiccant (such as e.g. bentonite clay, molecular sieves, or, preferably, silica gel) the shelf life of the packaged composition can be prolonged.

A suitable blister pack comprises or is formed of a top foil (which is breachable by the tablets) and a bottom part (which contains pockets for the tablets). The top foil may contain a metalic foil, particularly an aluminium or aluminium alloy foil (e.g. having a thickness of 20 μm to 45 μm, preferably 20 μm to 25 μm) that is coated with a heat-sealing polymer layer on its inner side (sealing side). The bottom part may contain a multi-layer polymer foil (such as e.g. poly(vinyl choride) (PVC) coated with poly(vinylidene choride) (PVDC); or a PVC foil laminated with poly(chlorotriflouroethylene) (PCTFE)) or a multi-layer polymer-metal-polymer foil (such as e.g. a cold-formable laminated PVC/aluminium/polyamide composition).

To ensure a long storage period especially under hot and wet climate conditions an additional overwrap or pouch made of a multi-layer polymer-metal-polymer foil (e.g. a laminated polyethylen/aluminium/polyester composition) may be used for the blister packs. Supplementary desiccant (such as e.g. bentonite clay, molecular sieves, or, preferably, silica gel) in this pouch package may prolong the shelf life even more under such harsh conditions.

The article may further comprise a label or package insert, which refer to instructions customarily included in commercial packages of therapeutic products, that may contain information about the indications, usage, dosage, administration, contraindications and/or warnings concerning the use of such therapeutic products. In one embodiment, the label or package inserts indicates that the composition can be used for any of the purposes described herein.

The present invention also provides methods particularly for treating metabolic diseases, by orally administering to a patient in need of such treatment one, two or more solid pharmaceutical dosage forms of the present invention. According to an embodiment the metabolic disease is type 2 diabetes mellitus. A preferred method according to this invention is an improvement of glycemic control in a patient with type 2 diabetes mellitus, in particular when treatment with empagilflozin, linagliptin, and metformin extended release is appropriate. The treatment is preferably an adjunct to diet and exercise.

Therefore another aspect of the present invention is the use of the solid pharmaceutical dosage form according to the present invention for the manufacture of a medicament for the treatment of a metabolic disease, in particular of type 2 diabetes mellitus.

Therefore another aspect of the present invention is the use of the solid pharmaceutical dosage form according to the present invention for the manufacture of a medicament for improving glycemic control in a patient with type 2 diabetes mellitus, in particular when treatment with empagilflozin, linagliptin, and metformin extended release is appropriate. The treatment is preferably an adjunct to diet and exercise.

Therefore another aspect of the present invention is the solid pharmaceutical dosage form according to the present invention for use in the treatment of a metabolic disease, in particular of type 2 diabetes mellitus.

Therefore another aspect of the present invention is the solid pharmaceutical dosage form according to the present invention for use in the improvement of glycemic control in a patient with type 2 diabetes mellitus, in particular when treatment with empagilflozin, linagliptin, and metformin extended release is appropriate. The treatment is preferably an adjunct to diet and exercise.

In one embodiment the patient in need of such treatment is a human, in particular an adult human. In an other embodiment the patient in need of such treatment is an adolescent human, i.e. a human of age 10 to 17 years or of age 13 to 17 years.

In one embodiment, the present invention provides a method for using a solid pharmaceutical dosage form according to the present invention in one or more of the following methods:
preventing, slowing the progression of, delaying or treating a metabolic disorder selected from the group consisting of type 1 or type 2 diabetes mellitus, impaired glucose tolerance, impaired fasting blood glucose, hyperglycemia, postprandial hyperglycemia, hyperinsulinemia and metabolic syndrome; or
slowing the progression of, delaying or treating of pre-diabetes; or
preventing, slowing the progression of, delaying or treating of an onset of type 2 diabetes mellitus; or
improving glycemic control and/or for reducing of fasting plasma glucose, of postprandial plasma glucose and/or of glycosylated hemoglobin HbA1c; or
preventing, slowing, delaying or reversing progression from impaired glucose tolerance, impaired fasting blood glucose, insulin resistance or from metabolic syndrome to type 2 diabetes mellitus; or
preventing, slowing the progression of, delaying or treating of a condition or disorder selected from the group consisting of complications of diabetes mellitus such as cataracts and micro- and macrovascular diseases, such as nephropathy, retinopathy, neuropathy, tissue ischaemia, diabetic foot, dyslipidemia, arteriosclerosis, myocardial infarction, accute coronary syndrome, unstable angina pectoris, stable angina pectoris, stroke, peripheral arterial occlusive disease, cardiomyopathy, heart failure, heart rhythm disorders and vascular restenosis; or
reducing body weight and/or body fat, or preventing an increase in body weight and/or body fat, or facilitating a reduction in body weight and/or body fat; or
preventing, slowing, delaying or treating the degeneration of pancreatic beta cells and/or the decline of the functionality of pancreatic beta cells and/or for improving and/or restoring the functionality of pancreatic beta cells and/or restoring the functionality of pancreatic insulin secretion; or
preventing, slowing, delaying or treating diseases or conditions attributed to an abnormal accumulation of ectopic fat, in particular liver fat; or
for maintaining and/or improving the insulin sensitivity and/or for treating or preventing hyperinsulinemia and/or insulin resistance;
in a patient with or at risk of oxidative stress, vascular stress and/or endothelial dysfunction, or diseases or conditions related or associated therewith, or
in a patient with or at risk of cardiovascular disease selected from myocardial infarction, stroke, peripheral arterial occlusive disease, or
in a patient with one or more cardiovascular risk factors selected from A), B), C) and D):
A) previous or existing vascular disease selected from myocardial infarction, coronary artery disease, percutaneous coronary intervention, coronary artery by-pass grafting, ischemic or hemorrhagic stroke, congestive heart failure, and peripheral occlusive arterial disease,
B) advanced age >/=60-70 years, and
C) one or more cardiovascular risk factors selected from
   advanced type 1 or type 2 diabetes mellitus >10 years duration,
   hypertension,
   current daily cigarette smoking,
   dyslipidemia,
   obesity,
   age >/=40
   metabolic syndrome, hyperinsulinemia or insulin resistance, and
   hyperuricemia, erectile dysfunction, polycystic ovary syndrome, sleep apnea, or family history of vascular disease or cardiomyopathy in first-degree relative;
D) one or more of the following:
   confirmed history of myocardial infarction,
   unstable angina with documented multivessel coronary disease or positive stress test,
   multivessel Percutaneous Coronary Intervention,
   multivessel Coronary Artery By-pass Grafting (CABG),
   history of ischemic or hemorrhagic stroke,
   peripheral occlusive arterial disease.
said method comprising administering one, two or more solid pharmaceutical dosage forms to the patient.

The present invention also relates to the use of the solid pharmaceutical dosage according to the present invention in the treatment and/or prevention of a cardiovascular disease in patients, for example in a type 1 or type 2 diabetes mellitus patient.

In another embodiment, the present invention provides a method of preventing, reducing the risk of or delaying the occurrence of a cardiovascular event in a patient with type 1 or type 2 diabetes mellitus or with pre-diabetes, in particular type 2 diabetes mellitus, said method comprising administering one, two or more solid pharmaceutical dosage forms according to the present invention to the patient. In one embodiment, the cardiovascular event is selected from cardiovascular death, non-fatal myocardial infarction, non-fatal stroke, hospitalisation for unstable angina pectoris and heart failure requiring hospitalisation. In one embodiment, the cardiovascular death is due to fatal myocardial infarction or fatal stroke. In one embodiment, the patient has or is at risk of a cardiovascular disease.

In one embodiment, the patient with type 1 or type 2 diabetes mellitus or with pre-diabetes has one or more cardiovascular risk factors selected from A), B), C) and D):
A) previous or existing vascular disease selected from myocardial infarction, coronary artery disease, percutaneous coronary intervention, coronary artery by-pass grafting, ischemic or hemorrhagic stroke, congestive heart failure, and peripheral occlusive arterial disease,
B) advanced age >/=60-70 years, and
C) one or more cardiovascular risk factors selected from
   advanced type 1 or 2 diabetes mellitus >10 years duration,
   hypertension,
   current daily cigarette smoking,
   dyslipidemia,
   obesity,
   age >/=40
   metabolic syndrome, hyperinsulinemia or insulin resistance, and
   hyperuricemia, erectile dysfunction, polycystic ovary syndrome, sleep apnea, or family history of vascular disease or cardiomyopathy in first-degree relative;
D) one or more of the following:
   confirmed history of myocardial infarction,
   unstable angina with documented multivessel coronary disease or positive stress test,
   multivessel Percutaneous Coronary Intervention,
   multivessel Coronary Artery By-pass Grafting (CABG),
   history of ischemic or hemorrhagic stroke,
   peripheral occlusive arterial disease.

Unless otherwise noted, the therapy according to the invention may refer to first line, second line or third line therapy, or initial or add-on combination therapy or replacement therapy.

According to an embodiment the solid pharmaceutical dosage form is provided for oral administration.

The solid pharmaceutical dosage form may be administered once-daily (QD) or twice-daily (BID), preferably once daily. For the once daily administration one or two solid pharmaceutical dosage forms may be administered to the patient. In the following Table 2 it is depicted which effective doses are achieved by once daily administration of one or two solid pharmaceutical dosage of different dose strengths to the patient.

TABLE 2

| | | | | |
|---|---|---|---|---|
| Solid pharmaceutical dosage form - | 1000/5/10 | 1000/5/25 | 1000/2.5/5 | 1000/2.5/12.5 |
| amount metformin hydrochloride/ | | | | |
| linagliptin/empagliflozin in mg | | | | |
| Administration once or twice daily | once daily | once daily | once daily | once daily |
| Number of solid pharmaceutical | 1 | 1 | 2 | 2 |
| dosage forms (tablets) per | | | | |
| admistration | | | | |
| effective administered dose | 1000/5/10 | 1000/5/25 | 2000/5/10 | 2000/5/25 |
| per day metformin hydrochloride/ | | | | |
| linagliptin/empagliflozin in mg | | | | |

Further, illustrative effects of the compositions according to the present invention are provided as follows, for example:

Several batches of solid pharmaceutical dosage forms (for example metformin hydrochloride/linagliptin/empagliflozin amounts are 1000 mg/5 mg/25 mg in a first set of batches and 1000 mg/2.5 mg/12.5 mg in a second set of batches) and different ratios (w/w) of linagliptin:arginine (for example 0:1, 2:1, 4:1 and 8:1) are stored open/closed at 40° C./75% r.h. over 3 months. Overall, under closed storage conditions, no significant change in linagliptin assay results in all batches are observed. Likewise, under closed storage conditions, no significant changes in linagliptin and metformin dissolution results are observed in the batches. Under open storage conditions, a correlation between arginine content and linagliptin assay results can be observed; based thereon, a linagliptin/arginine ratio of 1:4 (w/w) is selected as optimum.

In an embodiment, the solid pharmaceutical dosage forms of this invention preferably have dissolution properties such that, for example, after 2 hours 31-54% by weight of the metformin HCl active ingredient is dissolved, and/or after 4 hours 51-74% by weight of the metformin HCl active ingredient is dissolved, and/or after 12 hours not less than 80% by weight of the metformin HCl active ingredient is dissolved.

In a further embodiment, the pharmaceutical dosage forms of this invention preferably have dissolution properties such that after 45 minutes at least 75%, or at least 80%, or at least 90% by weight of linagliptin is dissolved. In a particular embodiment, after 30 minutes for linagliptin at least 80% (preferably not less than 75%) by weight of linagliptin is dissolved.

In a further embodiment, the pharmaceutical dosage forms of this invention preferably have dissolution properties such that after 45 minutes at least 75%, or at least 80%, or at least 90% by weight of empagliflozin is dissolved. In a particular embodiment, after 45 minutes for empagliflozin at least 80% (preferably not less than 75%) by weight of empagliflozin is dissolved.

The dissolution properties can be determined in standard dissolution tests, e.g. according to standard pharmacopeias (such as e.g. using paddle/basket method with agitation speed of 100 rpm, pH 6.8 buffer, and HPLC (linagliptin) and UV (metformin) analysis of the samples).

For illustrative example, in a certain embodiment, at pH 6.8 buffer the dissolution profiles of a solid pharmaceutical dosage form comprising a metformin hydrochloride extended release core, a barrier coat, a immediate release coating comprising linagliptin and empagliflozin and a color coat and a final coat according to this invention (such as e.g. according to the examples as in Table 3) and the respective reference innovator products (Jardiance®, Trajenta® and Glumetza®) are preferably similar (f2≥50) or in the same range, using e.g. the comparable innovator dissolution method.

### EXAMPLES

Solid pharmaceutical dosage forms comprising metformin HCl extended release, linagliptin and empagliflozin are film-coated tablets manufactured using typical processes and equipment for wet-granulation, tableting and film-coating. The core tablet contains metformin HCl for extended-release and may be based on an expandable polymeric swelling formulation that increases gastric retention and extends drug release from the matrix. This metformin HCl core tablet may be film-coated (spray-coated) with up to four layers (e.g. barrier coat layer, immediate-release active coat layer, color coat layer, final coat layer), one of which (active coat layer) contains the active pharmaceutical ingredients linagliptin and empagliflozin to add the immediate-release active components.

### Tablet Coating—Barrier Coat

Purified water is charged into a stainless steel mixing vessel equipped with a suitable mixer. While mixing at a speed that ensures vortex formation, the film forming system is added and mixed for at least 20 minutes. Mixing of the coating solution is continued throughout the coating operation. An appropriate quantity of metformin HCl extended release cores are charged to the coating pan of the perforated coating system equipped with a peristaltic pump and two spray nozzles. After the cores are preheated, pan rotation and spraying of the coating suspension begin. A fixed amount of the barrier coating suspension, which incorporates approximately 15% excess to account for losses during spraying, is applied to the cores. For example, the cores are coated to obtain a weight gain of 6%; to account for losses during spraying, an amount of coating solution equivalent to approximately 6.9% weight gain is used for spraying. Upon completion of the application of the coating solution, the cores are dried (target pan speed 3 rpm, target inlet air temperature 55° C.) for 15 minutes. The barrier-coated cores are then allowed to cool (product temperature not more than 35° C.).

Tablet Coating with linagliptin and empagliflozin

Purified water is charged into a stainless steel mixing vessel equipped with a suitable mixer. While mixing at a speed that ensures vortex formation, each component polyethylene glycol (PEG 6000), L-arginine, linagliptin, empagliflozin, talc and the Film Forming System (HPMC 2910 and PEG 8000) is added. The coating suspension is mixed for at least 20 minutes or until the last component added is homogenized. An appropriate quantity of barrier-coated tablet cores are charged to the coating pan of the perforated coating system equipped with a peristaltic pump and two spray nozzles. After the cores are preheated, pan rotation and spraying of the coating suspension begin. A fixed amount of the active coating suspension, which incorporates approximately 10% excess to account for losses during spraying, is applied. Alternatively the cores are coated to obtain a certain weight gain. The following table examplarily depicts certain weight gains for the various dose combinations and the ranges of weight gains used for spraying.

| | | | | |
|---|---|---|---|---|
| Linagliptin (mg) | 5 | 5 | 2.5 | 2.5 |
| Empagliflozin (mg) | 10 | 25 | 5 | 12.5 |
| Weight gain (% weight) | 6.25 | 7.25 | 5.09 | 5.59 |
| Range (% weight) | 5.63-6.88 | 6.53-7.98 | 4.58-5.60 | 5.03-6.15 |

Upon completion of the application of the coating solution, the cores are dried (target pan speed 3 rpm, target inlet air temperature 55° C.) for 15 minutes. The active-coated cores are then allowed to cool (product temperature not more than 35° C.).

The following Table 3 contains examples Ex.1 to 4 of solid pharmaceutical dosage forms according to the first aspect of the invention. In addition to the extended release core, the barrier coating and the immediate release coating, the solid pharmaceutical dosage forms comprise one or more color and/or final coatings which are not quantified in the Table 3.

TABLE 3
Example solid pharmaceutical dosage forms:

| | Ex. 1 | Ex. 2 | Ex. 3 | Ex. 4 |
|---|---|---|---|---|
| Extended release | | | | |
| core | | | | |
| Metformin | 1000 | 1000 | 1000 | 1000 |
| hydrochloride | | | | |
| Hydroxypropyl | 34.1 | 34.1 | 34.1 | 34.1 |
| methylcellulose | | | | |
| (e.g. 3 cP, HPMC | | | | |
| 2910-5) | | | | |
| Polyethylene oxide | 369.9 | 369.9 | 369.9 | 369.9 |
| (e.g. high molecular | | | | |
| weight, e.g. about | | | | |
| 7,000,000, such as | | | | |
| WSR-303 NF TG | | | | |
| LEO) | | | | |
| Magnesium stearate | 15 | 15 | 15 | 15 |
| Barrier coating | | | | |
| Titanium dioxide | 10.6 | 10.6 | 10.6 | 10.6 |
| Hydroxypropyl | 28.7 | 28.7 | 28.7 | 28.7 |
| methylcellulose | | | | |
| (e.g. HPMC 2910, 3 | | | | |
| cP) | | | | |
| Hydroxypropyl | 28.7 | 28.7 | 28.7 | 28.7 |
| cellulose | | | | |
| Talc | 17 | 17 | 17 | 17 |
| Immediate release | | | | |
| coating | | | | |
| Polyethylene glycol | 10 | 10 | 10 | 10 |
| (6000 USP/NF) | | | | |
| L-Arginine | 20 | 20 | 10 | 10 |
| Linagliptin | 5 | 5 | 2.5 | 2.5 |
| Empagliflozin | 10 | 25 | 5 | 12.5 |
| Talc | 9 | 9 | 9 | 9 |
| Hydroxypropyl- | 38 | 38 | 38 | 38 |
| methylcellulose | | | | |
| (HPMC 2910, 3 cP) | | | | |
| Polyethylene glycol | 2 | 2 | 2 | 2 |
| (8000 USP/NF) | | | | |
| Purified water | —* | —* | —* | —* |

*Processing agent water used in manufacturing and removed by drying

Optionally, the above compositions may be further coated by a color coat layer and/or a clear final coat. Typically, the color coat and the final coat may represent 1-4% w/w (preferably 1-3% w/w) of the total composition and each comprise one or more film-forming agents, one or more plasticizers, one or more optional glidants, and one or more optional pigments and/or colors. For example, a color coat may comprise hydroxypropyl methylcellulose (e.g. HPMC of 3 cP and/or HPMC of 6 cP), polyethylene glycol (e.g. PEG 8000), titanium dioxide and a color or lake. For example, a final coat may comprise hydroxypropyl methylcellulose (e.g. HPMC of 3 cP) and polyethylene glycol (e.g. PEG 8000) as well as a polishing wax (e.g. carnauba wax).

1. A solid pharmaceutical dosage form comprising in the order from the core to the outside
a) an extended release core comprising metformin hydrochloride and one or more excipients, and
b) one or more immediate release coatings comprising linagliptin and/or empagliflozin and one or more stabilizers for stabilizing linagliptin and one or more excipients,
wherein the amount of linagliptin is in the range from 2.5 mg to 5 mg, the amount of empagliflozin is in the range from 5 mg to 25 mg and the amount of metformin hydrochloride is in the range from 500 mg to 2000 mg.

2. The solid pharmaceutical dosage form of claim 1 further comprising one or more barrier coatings between the extended release core and the one or more immediate release coating(s).

3. The solid pharmaceutical dosage form of claim 1 further comprising one or more color and/or final coatings on the outside of the one or more immediate release coating(s).

4. A solid pharmaceutical dosage form according to claim 1 comprising in the order from the core to the outside
a) an extended release core comprising metformin hydrochloride and one or more excipients, and
b) none, one or more barrier coatings, and
c) an immediate release coating comprising linagliptin and empagliflozin and one or more stabilizers for stabilizing linagliptin and one or more excipients,
d) none, one or more color and/or final coatings,
wherein the amount of linagliptin is in the range from 2.5 mg to 5 mg, the amount of empagliflozin is in the range from 5 mg to 25 mg and the amount of metformin hydrochloride is in the range from 500 mg to 2000 mg.

5. A solid pharmaceutical dosage form according to claim 1 comprising in the order from the core to the outside
a) an extended release core comprising metformin hydrochloride and one or more excipients, and
b) none, one or more barrier coatings, and
c) a first immediate release coating comprising one of the active ingredients linagliptin or empagliflozin and in case the active ingredient is linagliptin one or more stabilizers for stabilizing linagliptin and one or more excipients, and
d) optionally a barrier coating, and
e) a second immediate release coating comprising the other one of the active ingredients empagliflozin or linagliptin and in case the active ingredient is linagliptin one or more stabilizers for stabilizing linagliptin and one or more excipients,
f) none, one or more color and/or final coatings,
wherein the amount of linagliptin is in the range from 2.5 mg to 5 mg, the amount of empagliflozin is in the range from 5 mg to 25 mg and the amount of metformin hydrochloride is in the range from 500 mg to 2000 mg.

6. The solid pharmaceutical dosage form according to claim 5 wherein the first immediate release coating comprises linagliptin and one or more stabilizers for stabilizing linagliptin and one or more excipients, and wherein the second immediate release coating comprises empagliflozin and one or more excipients.

7. The solid pharmaceutical dosage form according to claim 5, wherein there is a barrier coating between the first and second immediate release coating.

8. The solid pharmaceutical dosage form according to claim 1, wherein the extended release core is a formulation comprising metformin hydrochloride, a swellable and/or extended release material, and one or more further excipients.

9. The solid pharmaceutical dosage form according to claim 8 wherein the swellable and/or extended release material comprises polyethylene oxide and hydroxypropyl methylcellulose (hypromellose).

10. The solid pharmaceutical dosage form according to claim 4, wherein the barrier coating is each a film coat formulation independently comprising a film-coating agent, a plasticizer, and, optionally, a glidant, one or more pigments and/or colors.

11. The solid pharmaceutical dosage form according to claim 1, wherein the immediate release coating is a film coat formulation comprising linagliptin, empagliflozin, one or more stabilizers for stabilizing linagliptin, one or more film coating agents, one or more plasticizers and optionally one or more glidants.

12. The solid pharmaceutical dosage form according to claim 1, wherein the stabilizer in the immediate release coating is L-arginine.

13. The solid pharmaceutical dosage form according to claim 1, wherein the immediate release coating is a film coat formulation comprising linagliptin, empagliflozin, L-arginine as a stabilizer, a film coat formulation comprising hydroxypropylmethylcellulose and polyethylene glycol, polyethylene glycol as a plasticizer and optionally one or more glidants.

14. The solid pharmaceutical dosage form according to claim 12 wherein the weight ratio of the stabilizer L-arginine to linagliptin is within the range from about 8:1 to about 1:1, preferably from about 8:1 to about 2:1, more preferably from about 6:1 to about 2:1.

15. The solid pharmaceutical dosage form according to claim 3, wherein the color coating and final coating each independently of each other comprise a film coat formulation and one or more pigments.

16. The solid pharmaceutical dosage form according to claim 1, wherein the amount of linagliptin is 2.5 mg, the amount of empagliflozin is 5 mg or 12.5 mg and the amount of metformin hydrochloride is 1000 mg, or
wherein the amount of linagliptin is 5 mg, the amount of empagliflozin is 10 mg or 25 mg and of metformin hydrochloride is 1000 mg.

17. A method of treatment of type 2 diabetes mellitus in a patient in need thereof wherein a solid pharmaceutical dosage form according to claim 1 is orally adminstered to the patient.

18. The solid pharmaceutical dosage form according to claim 6, wherein there is a barrier coating between the first and second immediate release coating.

19. The solid pharmaceutical dosage form according to claim 13 wherein the weight ratio of the stabilizer L-arginine to linagliptin is within the range from about 8:1 to about 1:1, preferably from about 8:1 to about 2:1, more preferably from about 6:1 to about 2:1.
